(19) United States
(12) Patent Application Publication
(10) Pub. No.: US 2003/0026767 A1
(43) Pub. Date: Feb. 6, 2003

(54) Method to aerosolize Interferon-Gamma for lung delivery for local and systemic treatments

(76) Inventors: Nahed M. Moshen, Plymouth, MI (US); Thomas A. Armer, Cupertino, CA (US)

Correspondence Address: FITZPATRICK CELLA HARPER & SCINTO, 30 ROCKEFELLER PLAZA, NEW YORK, NY, 10112, US

(21) Appl. No.: 10/189,535

(22) Filed: Jul. 8, 2002

Related U.S. Application Data
(60) Provisional application No. 60/303,068, filed on Jul. 6, 2001.

(51) Int. Cl. A61L 9/04; A61K 38/21; B29B 9/00; A61K 9/14

(52) U.S. Cl. 424/46; 521/98; 424/85.5; 264/5; 530/351

(57) ABSTRACT

A method of making nanoparticles of interferon gamma for use in pharmaceutical formulations, and the treatment of various ailments by inhalation of these formulations are described. The interferon gamma is made into nanoparticle size using supercritical fluids technology.

This application claims benefit and incorporates by reference the entire disclosure of provisional application number 60/303,068 filed Jul. 6, 2001.

### BACKGROUND OF THE INVENTION

### FIELD OF THE INVENTION

The present invention provides a method to aerosolize natural or recombinants Interferon Gamma (IG) using propellant based delivery systems, dry powder delivery systems or solution, aqueous or dispersion based delivery systems.

IG is a naturally occurring protein, which can be recovered from naturally occurring sources or recombinant technology. It has been found to stimulate the immune system and to play a role in preventing the formation of excessive scar tissue. In its immune function role, IG is believed to directly induce the synthesis of enzymes which allow macrophages to kill phagocytosed microbes, resulting in the increased killing and removal of infectious organisms, among other cellular functions. This ability makes IG a potent antimicrobial agent.

Another function of IG is to help the body regulate the activity of fibroblasts. IG can help prevent excessive scarring by blocking the multiplication of fibroblasts and TGF-beta, a scar-inducing molecule.

With the identification of these intracellular functions, clinical use of IG for ailments such as difficult to treat mycobacterial disease (i.e., multi-drug resistant tuberculosis), chronic granulomatous disease- an immune deficiency based ailment, and osteopetrosis have been and are under consideration.

Attempts at clinical use of this potent agent have suffered from drawbacks, however. Very high doses, such as 500 μg of IG are being administered by injection or nebulizer. At such high doses, patients have demonstrated significant side effect and withdrawal problems. The necessity for such high doses also increases the costs of treatment.

In the present invention, the high doses used in existing clinical applications are significantly reduced by the aerosolization of the IG. When presented in this form, the IG can be administered in amounts as small as 5 μg, thus not only reducing the drug side effect, but also significantly reducing the cost of the treatment.

### DETAILED DESCRIPTION OF THE INVENTION

The method provides an IG with optimum particle design combined with an optimum delivery system to achieve efficient drug delivery to appropriate biospace in the respiratory tract (mouth, throat, lungs, alveoli) to treat local and systemic disorders. The method uses different particle design methods to achieve specific shapes, morphologies and sizes that function best with a particular delivery system to achieve optimum delivery. In addition, these different particle designs contribute to achieve desired pharmacokinetic and pharmacodynamic effects for the treatment of different disorders.

According to this invention, homologous, natural, or recombinant IG 1a or 2b can be made into particles with narrow particle size distribution with a mean volumetric diameter ranging from 50 nanometer to 3 micrometer. The particles are stable over time and temperature ranging from 5° C. to 40° C. without significant loss of bioactivity.

The IG particle diameter can be obtained by Supercritical Fluids (SF) processes, including Rapid Expansion of Supercritical Solutions (RESS), or Solution Enhanced Dispersion of Supercritical fluids (SEDS), as well as other techniques involving supercritical fluids. The use of SF process to form particles is reviewed in Palakodaty, S., et al., “Phase Behavioral Effects on Particle Formation Processes Using Supercritical Fluids”, Pharmaceutical Research, vol. 16. p. 976 (1999). These methods permit the formation of micron and sub-micron sized particles with differing morphologies depending on the method and parameters selected.

In addition, the IG particles used in the present invention can be fabricated by precipitation, cryogrinding, spray drying, micronization, lyophilization, volume exclusion, and any other methods of particle generation.

The objective of these processes is to create IG particles that are low in impurities, imperfections and surface charges and to thereby inhibit particle cohesion and agglomeration. Such particles more easily flow or disperse in fluid media including gases, vapors and liquids. In addition, processes such as SF result in increased IG purity, thus contributing to its enhanced activity. For example, SF techniques can result in separation of IG molecule fragments from intact IG molecules, thus contributing to enhanced activity and effectiveness of the IG, as well as the possibility of dose reduction. The processes can selectively remove low molecular weight and isomorphic or stereo isomer impurities.

Furthermore, these processes for producing micron sized particles, including SF, can permit selection of a desired morphology (e.g., amorphous, crystalline, resolved racemic) by appropriate adjustment of the conditions for particle formation during precipitation or condensation. As a consequence of selection of the desired particle form, extended release of the IG can be achieved. Also, fabricating IG into microspheres by volume exclusion induced precipitation can result in extended release profiles of the medicament to achieve specific pharmacokinetics and pharmacodynamic effects.

The method of this invention provides a IG formulation that can be delivered using propellant-based delivery systems. The formation is composed of (i) natural or recombinant IG, (ii) 1,1,1,2,3,3,3-heptafluoro-n-propane and/or 1,1,1,2-tetrafluorethane as propellants or any mixture of both with any proportions, (iii) with or without a surfactant and/or surface coating agent, and (iv) with and without trace amount of adjuvants. More specifically, the formulation consists of 0.02-0.06% w/w of IG as a medicament with a mean particle size of 1.5-5.0, um obtained by volume exclusion with 0.0001-0.002% w/w amounts of polymer, 99.8% w/w of 1,1,1,2,3,3,3-heptafluoro-n-propane and/or 1,1,1,2-tetrafluorethane as propellants or any mixture of both with any proportions, 0.005% to 0.02% w/w isopropyl myristate, lecithin, oleic acid, etc. as a surfactant surface coating agent, and 0.2-0.3% w/w ethanol as an adjuvant.

The formulation in this invention is packaged in aluminum-anodized canisters or polymer coated aluminum canisters crimped with a valve using known techniques.

The aerosol formulation in the present invention is manufactured by placing the medicament in a canister by lyophilization or simple placement. The surfactant with the adjuvant are mixed together and are transferred to the medicament in the canister. The canister is crimped with the valve, then propellant mixture is forced by pressure filling through the valve. The canister containing the aerosol formulation is then sonicated to assure thorough mixing and surfactantmedicament surface wetting.

This invention applies to any form of scale-ups employing cold and pressure filling. The adjuvant in the present invention is used to facilitate surfactant handling, while the surfactant in the present formulation invention is used to lubricate the valve and to facilitate the dispersibility of the medicament in the propellant.

The particles can be formulated into dry powder formats for use in unit or multidose dry powder inhalers or biphasic injection. The powder can be neat and mixed with a carrier or dispersed to aid in metering and delivery.

Regardless of the particle or powder form selected (i.e., crystalline, amorphous, rod-shaped, etc.), or the method selected to form the particles, the IG can be formed into nanoparticles and dispersed. Thus, IG can be fabricated into a nanopowder and then formulated into a liquid dispersion. Alternatively, IG can also be delivered by nebulizer in a solution or suspension. Also the IG formed into the powdered design selected can be blended, coated or let down by PEG, PVP, rHSA, starch, cyclodextrins, trihalose, lactose or sucrose thus permitting the IG to be loaded and metered into unit dose or multidose systems for dry powder inhalation delivery.

In any of the methods described above, the IG particles or dispersion formulations are stable, maintaining potency and biological activity for the intended use and storage life conditions.

This invention provides an effective aerosol formulation for delivery to the lung in order to treat idiopathic pulmonary fibrosis, chronic granulomatous disease, malignant osteopetrosis and multidrug-resistant tuberculosis therapies with a low amount of active ingredient. Said treatment can be either local or systemic. The dose of IG per puff can vary in a range from 5-200 itg, most preferably 5-100 ktg.

This invention also provides an aerosol formulation for the treatment of idiopathic pulmonary fibrosis, chronic granulomatous disease, malignant osteopetrosis and multidrug-resistant tuberculosis, cystic fibrosis using the pulmonary system as a route of administration for local treatment. 100231 Treatment with IG in accordance with this invention may be in conjunction with other suitable therapies.

What we claim is:

1. A method of forming natural interferon gamma into particles in the micron range.

2. A method of forming natural interferon gamma into nanoparticles.

3. An aerosol formulation for natural interferon gamma comprising interferon gamma and a propellant.

4. The formulation of claim 3 which further comprises a surfactant.

5. The formulation of claim 3 which further comprises an adjuvant.

6. An aerosolized formulation of natural interferon gamma comprising:
a. 0.02-0.06% w/w of natural interferon gamma having a mean particle size of 1.5-5.0 μm;
b. 0.0001-0.002% w/w polymer;
c. 99.8 w/w of 1,1,1,2,3,3,3-heptafluoro-n-propane and/or 1,1,1,2tetrafluorethane in any proportion;
d. 0.0005% to 0.2% w/w of a surfactant; and
e. 0.2-0.3% w/w ethanol.

7. An aerosol formulation comprising powdered interferon gamma with or without a carrier for use in dry powder inhalers.

8. The method according to claim 1 wherein the micron particles or nanoparticles are formed using supercritical fluids.

9. The method according to claim 8 wherein the method of forming nanoparticles is rapid expansion of supercritical solutions.

10. The method according to claim 8 wherein the method of forming nanoparticles is solution enhanced dispersion by supercritical fluids.

11. A method of treating disease in a human by delivery of natural interferon gamma to a human lung in an aerosol formulation.

12. The method of treating disease according to claim 11 wherein the disease is selected from the group consisting of idiopathic pulmonary fibrosis, chronic granulomatous disease, malignant osteopetrosis, cystic fibrosis and multi-drug resistant tuberculosis.

13. The method according to claim 11 wherein said treatment is local.

14. The method according to claim 11 wherein a puff of the aerosol formulation contains from 5-200 mg of interferon gamma.

15. The method according to claim 11 wherein said treatment is systemic.

16. The method of claim 1 wherein a let down agent can be used to meter the dose.

17. The method of claim 16 where the let down agent is starch or dextrose.

18. The method of claim I wherein the particles can be aerosolized using a dry powder device.

19. The method of claim 1 wherein the particles can be formulated into suspension or colloid dispersion to be atomized by nebulization.
